(19) United States
(12) Patent Application Publication
(10) Pub. No.: US 2007/0253903 A1
(43) Pub. Date: Nov. 1, 2007

(54) Method for (Two-Step) Dosing and Dosage Finding

(75) Inventors: Bernhard Knab, Kohl (DE); Jorg Lippert, Leverkusen (DE); Roland Loosen, Erftstadt (DE); Andreas Schuppert, Kurten (DE); Michael Sevestre, Leverkusen (DE); Juri Solodenko, Leverkusen (DE); Stefan Willmann, Dusseldorf (DE)

Correspondence Address: NORRIS, MCLAUGHLIN & MARCUS, PA, 875 THIRD AVENUE, 18TH FLOOR, NEW YORK, NY, 10022, US

(73) Assignee: BAYER TECHNOLOGY SERVICES GMBH, LEVERKUSEN, GERMANY (DE)

(21) Appl. No.: 11/569,449

(22) PCT Filed: May 14, 2005
(86) PCT No.: PCT/EP05/05315

§ 371 Date: Jan. 4, 2007

(30) Foreign Application Priority Data
May 25, 2004 (DE) 10-2004-02534.2

(51) Int. Cl. A61K 49/00

(52) U.S. Cl. 424/9.1

(57) ABSTRACT

The invention relates to a method for dosing specific doses and timed dosage profiles of medicaments (in animals and humans) as well as agrochemicals (for the treatment of plants).

The invention relates to a method for dosing specific doses and timed dosage profiles of medicaments (in animals and humans) as well as agrochemicals (in the treatment of plants).

Besides using the correct active agent or the correct active agent combination, the success of medicament-based therapies or active agent application in agriculture depends crucially on selecting a suitable dose or a suitable dosage scheme, i.e. a timed dosage sequence. That dosage scheme which has the best benefit/risk ratio can be regarded as optimal. It maximizes the desired action while simultaneously minimizing the undesired side effects.

Conventional methods for determining dosages are based on empirical studies into the dose-action relationship of medicaments. Adaptation to the particular features of individual patients is generally done—if at all—empirically or on the basis of heuristics, for example allometric scaling. An improved predictive method for dosage calculation and application, which can take into account anatomical, physiological or genetic differences between individual bodies, is described in DE A 10 345 837 (Pharmacogenomics) and DE A 102004010516.2 (Dosage Device, Bayer). In both of these applications, the focus is on optimizing the pharmacokinetic profile. In many cases relevant to clinical therapy, however, the concentration-time relationship of the active agent at the action site is not on its own predictive for the success of the therapy since the therapeutic effect (or undesired side effects) is determined by the complex kinetics and dynamics of biochemical processes. Without a detailed knowledge of the action and side effect mechanisms, no meaningful therapy optimization can therefore be carried out.

The biological effect of an active agent and other chemical substances is determined by the time response of the substance concentration at the action site and the biochemical interactions at the action site. Prediction of actions is therefore possible only when predictive models of the substance absorption, distribution, metabolism and excretion (so-called ADME models) can predict the concentration at arbitrary places in a body, in combination with models of the biochemical action mechanism which can describe or predict the effect of a chemical substance in the body.

ADME models for a very wide variety of organisms (particularly humans and mammals such as apes, dogs, cats, rats, mice as well as invertebrates such as insects or crustaceans and a range of plant species) are known and prior art. Physiology-based pharmacokinetic models (so-called PBPK models) are of particular interest for this invention; these can describe and predict the ADME time response of substances in a body with the aid of compartment models and differential equation systems, and are likewise prior art (S. Willmann, J. Lippert, M. Sevestre, J. Solodenko, F. Fois, W. Schmitt: “PK-Sim®: a physiologically based pharmacokinetic ‘whole-body’ model”, Biosilico 1, 121-124 2003; P. S. Price, R. B. Conolly, C. F. Chaisson, E. A. Gross, J. S. Young, E. T. Mathis, D. R. Tedder: “Modeling interindividual variation in physiological factors used in PBPK models of humans”, Crit. Rev. Toxicol. 33, 469-503, 2003).

Models for predicting the effect of a chemical substance at an action site are likewise known and prior art. Besides expert systems which represent empirically obtained knowledge and make it usable for predictions, models for the dynamic simulation of metabolic networks and signal transduction networks are of particular interest for the present invention. Also interesting and particularly useful are models of the binding relationship of chemical substances with the body's own molecules, for example transport proteins such as PGP or enzymes such as the P450 cytochrome family, which play a crucial role for distribution in the body and biotransformation and therefore the breakdown of molecules.

Besides the great technical demands on the model formulation, complete integration of these model types (see FIGS. 1, 1.2) leads to model complexities which are difficult to handle numerically and—in practice—unfeasible for numerical optimization (schematic representation of the general optimization task in FIG. 1). This hurdle has hitherto prevented the integrated use of predictive models of the ADME response and the effect of chemical substances.

Owing to the complexity, none of the known methods gives satisfactory solutions.

On the basis of the prior art it is therefore an object to provide a method which can cope with the complexity of the processes, with the aim of making it possible to combine the optimal action simultaneously with minimal side effects. Such a method then also makes it possible to estimate the upper limits and tolerance values for exposure to poisonous substances.

The predictive method for determining optimal dosage, as described in the present application, is capable of taking into account individual differences in the pharmacokinetic and pharmacodynamic response of an administered substance between particular individuals. The latter is achieved by models for predicting the effect of a chemical substance at its action site. The method can be used directly when planning clinical studies. Besides improving the benefit/risk ratio for the individual subjects, the number of clinical studies and their duration can thereby be reduced and the likelihood of a successful study result can at the same time be increased considerably.

The method can likewise be used for individualized optimization of therapies in clinical practice. Besides an improvement of the healing process, using the method can also be expected to reduce costs for the medical treatment and shorten illness times.

Through the use of suitable biological models, the method can be used both for veterinary applications and for agrochemical issues (in the treatment of plants).

Since toxic effects can likewise be regarded as an (in this case undesired) action of chemical substances, the method is also capable of providing estimates of maximal exposures (doses and exposure times) for poisonous substances. These can be used in the scope of approving chemicals to plan experimental studies and for securing the evaluation of experimental findings.

The present invention is based on overcoming the complexity problem due to integration, by substantially separating the two model components by means of an iterative calculation of the concentration and action profiles of administered substances. By reversing the causal chain (an active agent is administered, is subsequently found in a particular concentration at the action site and consequently exerts its action), the complex optimization problem of determining dosages, in order to obtain a desired effect, is broken down into two simpler optimization steps which can be handled computationally (see FIG. 2):
Step 1 Determining one or more suitable concentration-time profiles, ideally the optimal concentration-time profile, for one or more substances at one or more action sites in order to achieve as great as possible a match with the desired effects. In the case of a plurality of substances or a plurality of action sites or a plurality of effects at an action site, optimal concentrations of one or more substances must be determined for each effect. This optimization step is performed with one or more predictive biological action models, which may be coupled together. The effects may be optimized either in a common optimization process or independently of one another (see FIGS. 2.1, 3, 4, 5, 6).
Step 2 Determining an optimal dosage for one or more substances in order to obtain as great as possible a match with the optimal concentration-time profiles which were determined in Step 1. This optimization step is performed with one or more detailed ADME models (for example PBPK models), which may be coupled together. The optimization may be performed independently of one another for each of the models or in a common optimization process (see FIGS. 2.2, 7, 8, 9, 10).

Following the two-stage optimization method, the dosage profiles obtained in this way are administered either manually or with the aid of a dosing device. All ways of administering active agents may be envisaged in the scope of manual dosage. In medical applications, depending on the application, this may involve giving tablets or capsules or suppositories, applying ointments and other suspensions, inhaling aerosols or gases, injecting solutions or administering such solutions by means of a drop. These types of administration may be envisaged both for humans and for animals. For the latter, it is possible to mix the active agents with animal food. In the case of fish, the active agent may be added to the water of an aquarium or another container which holds the one or more fish. The term dosing devices means all apparatus for which a dosing profile can be specified, either as a constant dosage value or as a time-variable dosing profile. Infusion machines, in particular, may be envisaged for medical applications. Besides this, technical devices for enriching inhaled air with a gas or aerosol are conceivable. In veterinary applications, this may moreover involve machines which perform automatic dosage of food or which add an active agent to the water of a fish aquarium or pond. In crop protection applications, besides manual methods for the dosage in crop protection applications, it is possible to use all ways of applying crop protection means including automatic spray machines for mobile as well as stationary use in glasshouses or on fields.

The method is suitable by design for handling the simultaneous administration of a plurality of active agents which interact in their pharmacokinetic behavior and their action, and the simultaneous observation of (desired) actions and (undesired) side effects. With this method, furthermore, it is readily possible to handle one or more active or inactive starting substances (prodrugs), which are converted into one or more active substances (metabolites) by metabolism in the body.

Since both the desired action of a medicament and the maximally tolerated undesired side effects of an active agent or any other substance (for example an environmental chemical or a food additive), or a combination of the two, is understood to be an effect in the context of the method, limit-value exposures may also be calculated besides the dosage scheme.

A schematic representation of the method according to the invention (in its simplest form) is shown in FIG. 2. The optimization of the local concentration of a substance as carried out in Step 1 is represented in the left-hand part of the FIG. (2.1). The optimization of the dosage of the substance as carried out in Step 2 is represented in the right-hand part of the FIG. (2.2).

The method begins with a freely selectable starting concentration-time profile for the active agent in question at the action site (FIGS. 2, 2.3), which is used as an input function for the biological effect model (FIGS. 2, 2.4). The biological action model may be adapted to parameters which have been obtained by means of technical diagnostic methods and are characteristic either of the indication or of the individual patient or body. The technical diagnostic methods used may be any biological, biometric, chemical or physical methods which are capable of determining model parameters; for example, information obtained by a biopsy about a tumor type from which a patient is suffering may be used in order to individualize the effect model for this patient. Furthermore, for example, information which has been determined by imaging methods about the size and morphology of a tumor may be used for the individualization. Another possible variant of the method is to obtain model parameters by means of literature research, and in particular with bioinformatic tools for searching in literature, chemistry, genetics, protein or signal transduction network databases. With the aid of this method, it is possible to find free parameters of the model which should not or cannot be individualized. The effect model then calculates the effect caused by the predetermined concentration profile (FIGS. 2, 2.5). In the next step, this is compared with a target effect specified by the indication (FIGS. 2, 2.6). If the target effect and the actual effect match, or if the deviation between the two does not exceed a threshold which is either predetermined (for example given by biological constraints) or determined by the optimization method (by a numerical criterion), then the concentration-time profile used as the input function in 2.3 is kept as a target concentration-time profile (FIGS. 2, 2.8) and Step 1 (FIG. 2, 2.1) is ended. The deviation between the two may be quantified by a suitable measure. This measure may for example be a continuous quantity e.g. a squared difference, or for example a discrete quantity e.g. the number of violations of a criterion. If there is a deviation between the actual and target effects in 2.6, then an optimization step is executed (FIGSS. 2, 2.7) in which the input profile (FIGS. 2, 2.3) is modified. All known numerical and analytical optimization methods may be envisaged as methods for carrying out the optimization. Especially gradient methods (for example Newton or quasi-Newton methods) among the numerical methods, or gradient-free methods (for example nested intervals), or stochastic methods (for example Monte-Carlo methods) or evolutionary methods (for example genetic optimization) are of particular interest. The particular embodiment of an analytical optimization method may be dictated by the effect model type used. All the individual steps are repeated iteratively until a match between the target effect and the actual effect is achieved in 2.6, and Step 1 can be terminated in 2.8.

Through the selection of the target effect, the deviation measure and the termination criterion for the comparison with the actual effect (FIGS. 2, 2.6), both actions and side effects (i.e. including toxicity) can be handled, for example by defining upper limits and establishing that not exceeding them is a termination criterion.

The target concentration-time profile (2.8) obtained in the first step is used in the second step (FIGS. 2, 2.2) as a target profile for the optimization of the dosage scheme (FIGS. 2, 2.9). Step 2 begins with a freely selectable starting dosage scheme (FIGS. 2, 2.9). With the aid of the ADME model (FIGS. 2, 2.10), for example a PBPK model, the concentration-time profile resulting at the action site from this dosage scheme is calculated (FIGS. 2, 2.11). The ADME model may be adapted and individualized with the aid of information about the indication and the active agent, as well as with physiological, anatomical or genetic properties of the individual patient or body. In a PBPK model, for example, adaptations could be performed for body size, body weight and body mass index. Information about the type (for example superficial, infiltrating, encapsulated), position and size of the tumor which is intended to be the action site of the treatment could likewise be used, for example if they have been obtained by imaging methods. If information is available for example about the patient's genotype, which influences for example the expression of transport proteins, then this could also be used for the individualization. Furthermore, it is possible to use any technical diagnostic methods which are capable of determining model parameters, i.e. all biological, biometric, chemical or physical and analysis processes and methods. Another possible variant of the method is to obtain model parameters by means of literature research, and in particular with bioinformatic tools for searching in literature, chemistry, genetics, protein or signal transduction network databases. With the aid of this method, it is possible to find free parameters of the model which should not or cannot be individualized.

The concentration-time profile at the action site, which is obtained in 2.11, is then compared with the target profile obtained in Step 1 (FIGS. 2, 2.12). If the target concentration-time profile and the actual concentration-time profile match, or if the deviation between the two does not exceed a threshold which is either predetermined or determined by the optimization method, then the dosage scheme used as the input function in 2.9 is kept as an optimized dosage scheme (FIGS. 2, 2.14) and Step 2 (FIGS. 2, 2.2) and the method is therefore ended. The deviation between the two may be quantified by a suitable measure. This measure may for example be a continuous quantity e.g. a squared difference, or for example a discrete quantity e.g. the number of violations of a criterion. If there is a deviation between the actual and target concentration-time profiles in 2.11, then an optimization step is executed (FIGS. 2, 2.13) in which the input dosage scheme (FIGS. 2, 2.9) is modified. All known numerical and analytical optimization methods may be envisaged as methods for carrying out the optimization. Especially gradient methods (for example Newton or quasi-Newton methods) among the numerical methods, or gradient-free methods (for example nested intervals), or stochastic methods (for example Monte-Carlo methods) or evolutionary methods (for example genetic optimization) are of particular interest. The particular embodiment of an analytical optimization method may be dictated by the ADME model type used. All the individual steps are repeated iteratively until a match between the target effect and the actual effect is achieved in 2.6, and Step 1 can be terminated in 2.8.

A variant of the method makes it possible to handle a plurality of effects (for example action and side effect) which are caused by an active agent or a substance at an action site (FIG. 3). The effect model in FIG. 2.4 is replaced by an arbitrary number (1 to N) of effect models for this action site (FIG. 3, 3.2). The effects calculated by these models (FIGS. 3, 3.3; 1 to N) are compared with a series of target effects, and the entire optimization method is carried out repeatedly from Step 1.

In a further variant, the method can be carried out both on a plurality of active agents and a plurality of action sites with a plurality of effects and arbitrary combinations of active agents, action sites and effects (FIG. 4). A plurality of concentration-time profiles at one or more action sites for one or more active agents or substances are now used as the input and the starting values (FIGS. 4, 4.1). In analogy with the procedure described above, a plurality of target concentration-time profiles are calculated in this case (FIGS. 4, 4.6).

A particular variant of the method outlined in FIG. 4 involves interactions and coupling of the effects of a plurality of active agents or a plurality of effects at one or more action sites (FIG. 5). In this case, the group of effect models in 4.2 must be replaced by an integrated effect model (FIGS. 5, 5.2). All the other substeps remain unchanged. The modified demands on the optimization method (FIGS. 5, 5.5) follow naturally. It should be noted that interactions between various substances can also influence their ADME response. The way in which to handle such coupling in the ADME response will be described after the variants of the method for handling coupled effects (see below).

The procedure described in FIG. 6 serves as a particular (simplified) variant of the method for a plurality of action sites (with one or more effects and one or more active agents or substances). Instead of simultaneously optimizing all the effects (as described above, FIGS. 3, 4 and 5), they are optimized independently of one another.

The variants of Step 1 of the method as described in FIGS. 4, 5 and 6 require variants for Step 2 of the method, which differ from that described in FIG. 2.

For the case of one active agent but a plurality of action sites, the comparison must be performed with a plurality of target profiles as described in FIG. 7.

For the case of a plurality of active agents, the ADME model in FIG. 2 (2.10) or FIG. 7 (7.2) must be replaced by a series of ADME models for each individual active agent.

If there are interactions between the ADME responses of a plurality of active agents, then the ADME models in FIG. 8 (8.2) must be replaced by an integrated ADME model (FIG. 9, 9.2).

In this case, it is also possible to handle administering/receiving one or more substances via a plurality of application paths, for example orally, intravenously, intra-arterially, intramuscularly, dermally, inhalatively or topically.

The procedure described in FIG. 10 serves as a particular (simplified) variant of the method for a plurality of active agents (with one or more effects and one or more active agents or substances) which do not interact in their ADME response. Instead of simultaneously optimizing all the concentration-time profiles (as described above, FIGS. 7, 8 and 9), they are optimized independently of one another.

In principle, all the methods based on the said parameters are suitable as ADME models, the method of PBPK modeling as claimed in DE A 10160270 and DE A 10345836 being particularly suitable and preferred according to the invention.

Besides using the method to determine an optimal dosage with the aim of achieving a concentration-time profile at the action site or action sites as determined in Step 1 of the method (FIGS. 2.1, 3, 4, 5, 6), variants of the method are also conceivable in which pharmacokinetic quantities derived from concentration-time profiles are the target function in Step 2 of the method. These derived pharmacokinetic quantities include for example maximal concentration, integrals of concentration-time curves, half-lives, mean residence times and periods of exceeding a threshold.

Besides the application of the method according to the invention as an aid for carrying out a medical therapy, the method according to the invention may also be used directly in clinical trials or animal trials, for example in order to start off the runs with clinically “sensible” dosages and to minimize the typical “settling in” of the dosage, i.e. the empirical-iterative arrival at excessive or insufficient doses which alternatingly approach the optimum, and therefore minimize the burden on the bodies being treated and maximize the likelihood of the experiment's success.

Humans, animals and plants are therefore suitable as a target group for the application of the method according to the invention, i.e. a body for which the method can be carried out, especially humans and economically useful, breeding, laboratory, test and pet animals. The method is particularly preferably used as an aid for the therapeutic treatment of humans or clinical trials on humans.

Economically useful and breeding animals include mammals, for example cows, horses, sheep, pigs, goats, camels, water buffalo, donkeys, rabbits, fallow deer, reindeer, animals prized for fur, for example mink, chinchillas, raccoons, birds, for example chickens, geese, turkeys, ducks, pigeons, bird species to be kept at home and in zoos.

Laboratory and test animals include mice, rats, guinea pigs, hamsters, rabbits, dogs, cats, pigs and apes, respectively in all species, subspecies and breeds.

Pet animals include dogs, cats, birds and fish.

Studies to estimate the toxicity and maximal exposures to a substance respectively represent a preferred application of the method.

The method according to the invention is particularly advantageous for medical applications, and especially those indications and active agents which have only a narrow “therapeutic window”. A narrow therapeutic window means that there is only a small concentration range in which the desired pharmacological effects do actually occur but at the same time no undesired side effects are to be observed. Examples of indication fields are all types of cancer diseases, infectious diseases, in particular bacterial and viral infections, cardiovascular diseases, in particular high blood pressure, lipidemia, angina pectoris and myocardial infarction, diseases of the central nervous system such as Alzheimer's disease, schizophrenia, epilepsy, chronic headaches (migraines), analgesia and anesthesia, psychiatric diseases, in particular depression and anxiety, metabolic diseases, for example diabetes and impairments of fat metabolism (obesity), respiratory diseases such as asthma and bronchitis, immune diseases, in particular allergies, rheumatism and multiple sclerosis, diseases of the gastrointestinal tract, in particular ulcers of the stomach and duodenum and Crohn's disease, as well as vascular diseases, in particular those which cause erectile dysfunction, and states of acute shock.

### DESCRIPTION OF THE FIGURES

FIG. 1: schematic representation of the general optimization problem for predicting an optimal dosage of active agents.

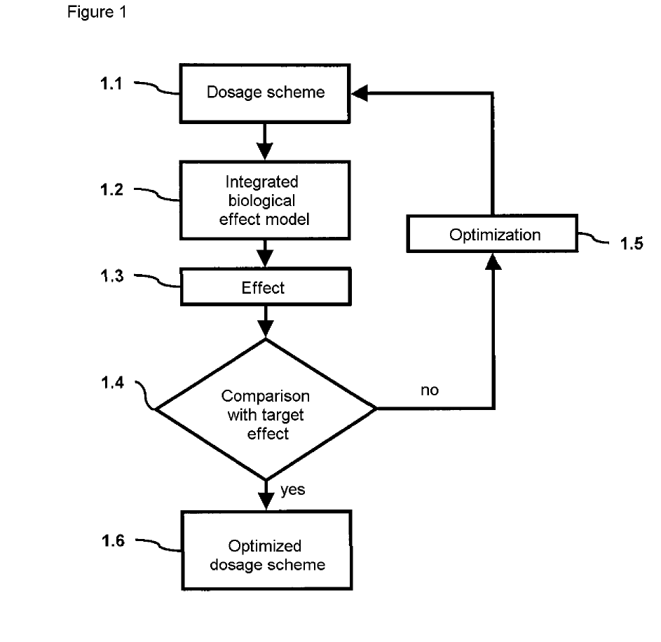

FIG. 2: schematic representation of the two-stage method for dose and dosage determination.

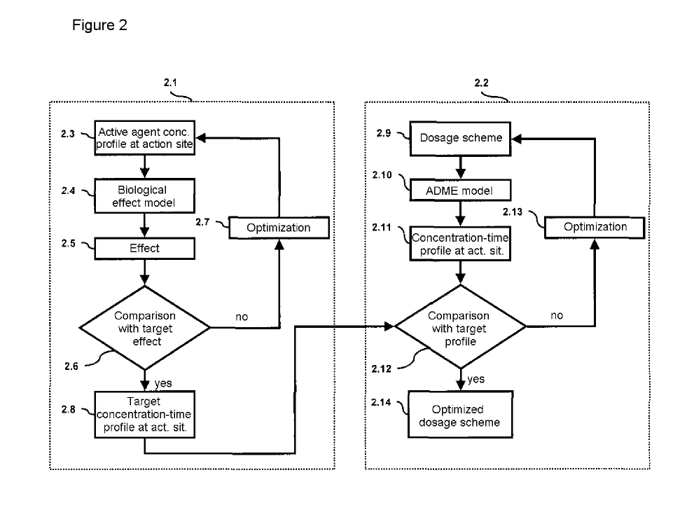

FIG. 3: schematic representation of Step 1 of the two-stage method for dose and dosage determination for a plurality of effects at one action site.

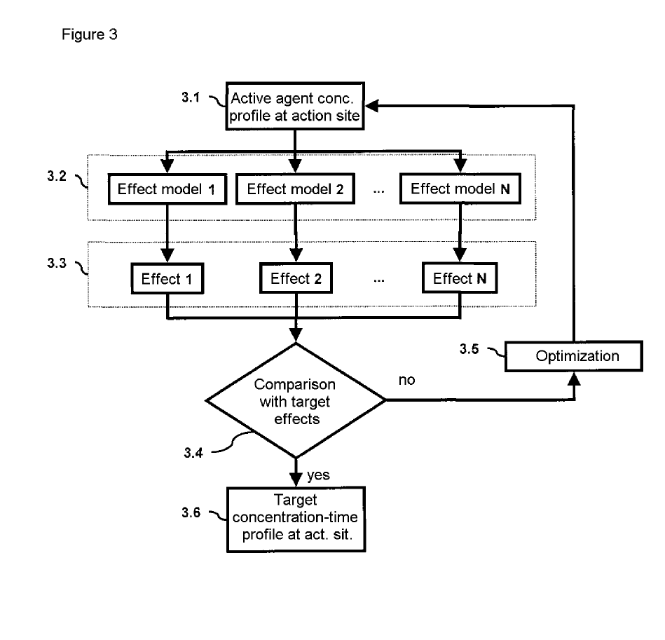

FIG. 4: schematic representation of Step 1 of the two-stage method for dose and dosage determination for a plurality of effects and/or active agents and/or action sites.

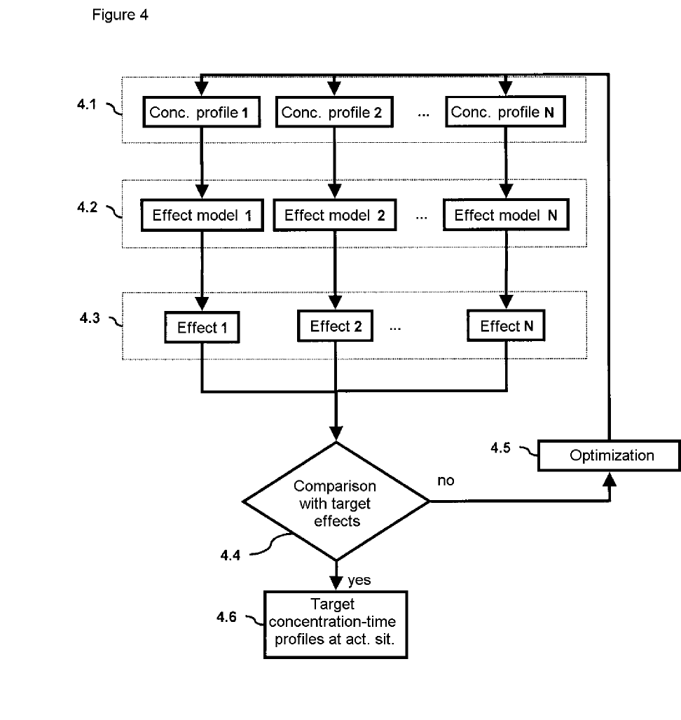

FIG. 5: schematic representation of Step 1 of the two-stage method for dose and dosage determination for a plurality of effects and/or active agents and/or action sites with coupling and interactions between the effects, active agents and action sites.

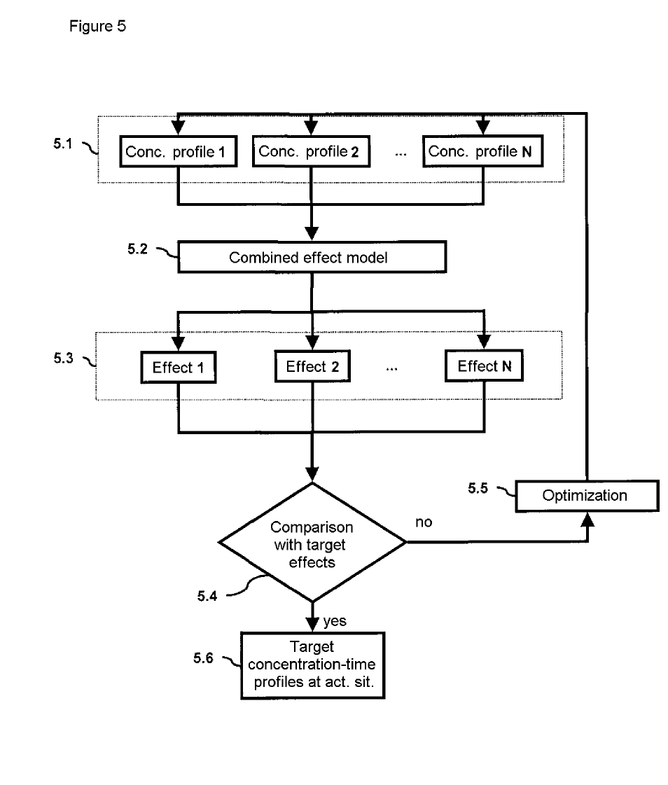

FIG. 6: schematic representation of Step 1 of the simplified two-stage method for dose and dosage determination in the absence of coupling.

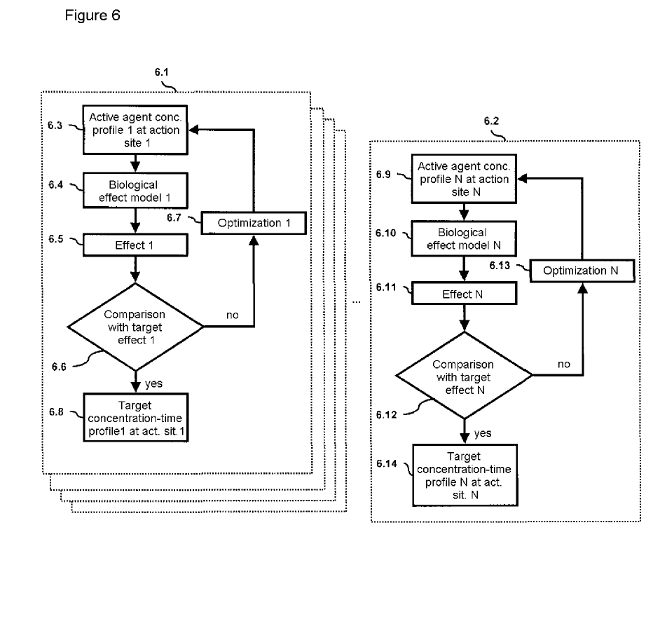

FIG. 7: schematic representation of Step 2 of the method for the timed dosage of medicaments for a plurality of action sites.

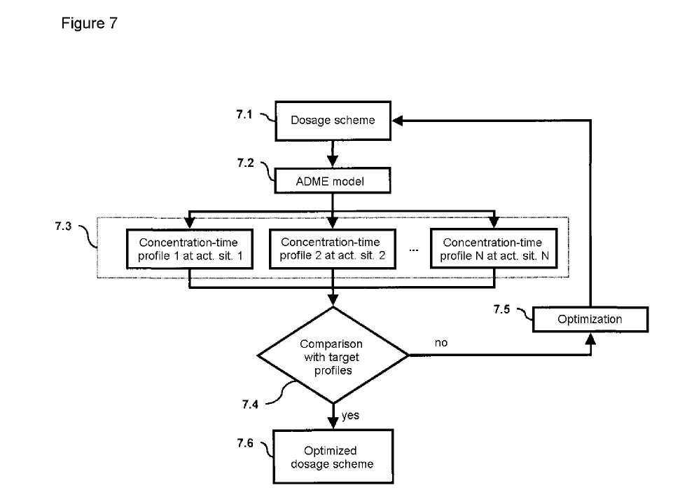

FIG. 8: schematic representation of Step 2 of the method for the timed dosage of medicaments for a plurality of active agents and/or action sites.

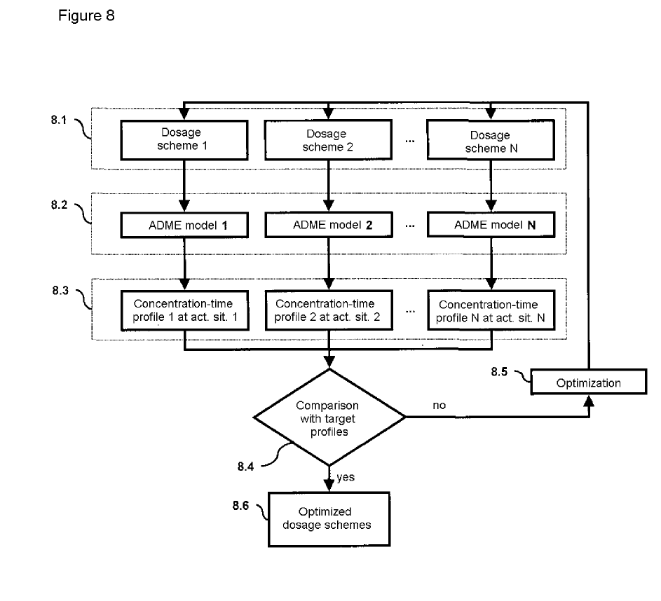

FIG. 9: schematic representation of Step 2 of the method for the timed dosage of medicaments for a plurality of active agents and/or action sites and/or application types and in the presence of interactions between the ADME responses.

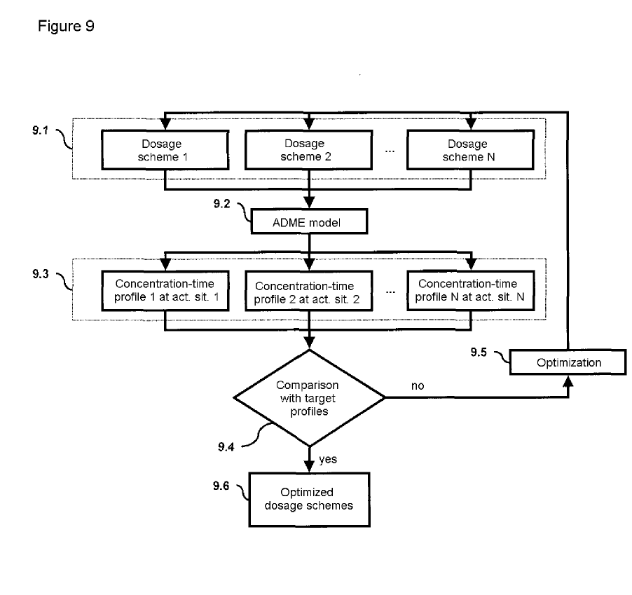

FIG. 10: schematic representation of Step 2 of the simplified method for the timed dosage of medicaments in the absence of coupling and interactions.

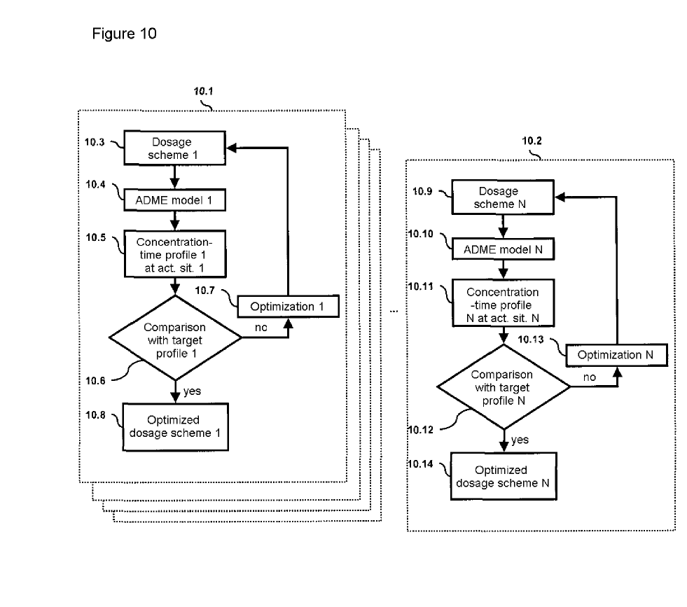

1. A method for determining and administering doses and timed dosage profiles, wherein a suitable concentration-time profile for a desired treatment is determined in a first step by an approximation method, an optimal dosage necessary for this is determined in a second step by a further approximation method and the dosage determined in this way is prepared for application or correspondingly applied.

2. The method as claimed in claim 1, wherein an ADME model is used for determining the dosage.

3. The method as claimed in claim 1, wherein a cellular action model is used for determining the concentration-time profile.

4. The method as claimed in claim 1, wherein a data-driven modeling method is used for determining the concentration-time profile or the dosage.

5. The method as claimed in claim 1, which is used in order to perform dosages in medical or veterinary applications.

6. The method as claimed in claim 1, which is used in order to perform dosages in crop protection applications.

7. The method as claimed in claim 1, which is used in order to perform toxicity estimates or carry out risk assessments by optimizing in respect of maximally tolerable actions instead of optimal actions.

8. The method as claimed in claim 1, wherein one of the following numerical optimization methods is employed as an approximation method: gradient methods; gradient-free methods; and stochastic methods.

9. The method as claimed in claim 1, wherein the dosing of the active agent takes places manually or by a suitable device.

10. The method as claimed in claim 1, wherein parameters necessary for the parameterization are obtained by means of biological, biometric, chemical or physical methods for the model.
